(19) United States
(12) Patent Application Publication
(10) Pub. No.: US 2007/0148244 A1
(43) Pub. Date: Jun. 28, 2007

(54) Drug delivery systems

(76) Inventors: Jay F. Kunzler, Canandaigua, NY (US); Joseph C. Salamone, Boca Raton, FL (US)

Correspondence Address: Bausch & Lomb Incorporated, One Bausch & Lomb Place, Rochester, NY, 14604-2701, US

(21) Appl. No.: 11/316,592

(22) Filed: Dec. 22, 2005

(51) Int. Cl. A61K 9/14

(52) U.S. Cl. 424/486

(57) ABSTRACT

Matrix controlled diffusion drug delivery systems based on polymerization products of monomeric mixture comprising (a) one or more silicone-containing monomers of the general formula: 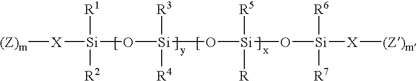
wherein x, y, m, m′, R, R¹, R², R³, R⁴, R⁵, R⁶, R⁷, X, Z and Z′ are as defined herein and (b) silicon hydride-containing monomers are provided, wherein the matrix controlled diffusion drug delivery systems are sized and configured for back of the eye delivery.

### BACKGROUND OF THE INVENTION

1. Technical Field

The present invention relates generally to drug delivery systems, and methods of treatment.

2. Description of Related Art

Conventional drug delivery involving frequent periodic dosing is not ideal or practical in many instances. For example, with more toxic drugs, conventional periodic dosing can result in high initial drug levels at the time of dosing, followed by low drug levels between doses often times below levels of therapeutic value. Likewise, conventional periodic dosing may not be practical or therapeutically effective in certain instances such as with pharmaceutical therapies targeting areas of the inner eye or brain in need of treatment such as the retina.

During the last two decades, significant advances have been made in the design of controlled release drug delivery systems. See, e.g., U.S. Patent Application Publication Nos. 2004/0043067 and 2004/0253293. Such advances have been made in an attempt to overcome some of the drug delivery shortcomings noted above. In general, controlled release drug delivery systems include both sustained drug delivery systems designed to deliver a drug for a predetermined period of time, and targeted drug delivery systems designed to deliver a drug to a specific area or organ of the body. Sustained and/or targeted controlled release drug delivery systems may vary considerably by mode of drug release within three basic drug controlled release categories. Basic drug controlled release categories include diffusion controlled release, chemical erosion controlled release and solvent activation controlled release. In a diffusion controlled release drug delivery system, a drug is surrounded by an inert barrier and diffuses from an inner reservoir, or a drug is dispersed throughout a polymer and diffuses from the polymer matrix. In a chemical erosion controlled release drug delivery system, a drug is uniformly distributed throughout a biodegradable polymer. The biodegradable polymer is designed to degrade as a result of hydrolysis to then uniformly release the drug. In a solvent activation controlled release drug delivery system, a drug is immobilized on polymers within a drug delivery system. Upon solvent activation, the solvent sensitive polymer degrades or swells to release the drug. Unfortunately, controlled release drug delivery systems to date do not provide a means by which one may manipulate and control drug delivery systems' drug release rate for specific drugs over a broad range of drugs.

Because of the noted shortcomings of current controlled release drug delivery systems, a need exists for controlled release drug delivery systems that allow for manipulation and control of drug release rates depending on the drug to be delivered, the location of delivery, the purpose of delivery and/or the therapeutic requirements of the individual patient.

### SUMMARY OF THE INVENTION

In accordance with one embodiment of the present invention, a matrix controlled diffusion drug delivery system is provided comprising a therapeutically effective amount of one or more pharmaceutically active agents entrapped in a polymerization product of a monomeric mixture comprising (a) one or more silicone-containing monomers of the general formula: 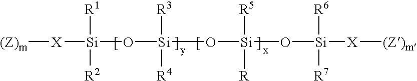
wherein x, y, m, m′, R, R¹, R², R³, R⁴, R⁵, R⁶, R⁷, X, Z and Z′ are as defined herein and (b) one or more silicon hydride-containing monomers, wherein the matrix controlled diffusion drug delivery system is sized and configured for back of the eye delivery.

In accordance with a second embodiment of the present invention, a process for preparing a matrix controlled diffusion drug delivery system sized and configured for back of the eye delivery is provided comprising the steps of entrapping a therapeutically effective amount of one or more pharmaceutically active agents in a polymerization product of a monomeric mixture comprising (a) one or more silicone-containing monomers of the general formula: 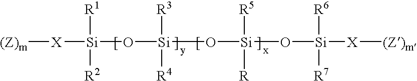
wherein x, y, m, m′, R, R¹, R², R³, R⁴, R⁵, R⁶, R⁷, X, Z and Z′ are as defined herein and (b) one or more silicon hydride-containing monomers.

In accordance with a third embodiment of the present invention, a process for preparing a matrix controlled diffusion drug delivery system sized and configured for back of the eye delivery is provided, the process comprising (a) swelling a polymerization product of a monomeric mixture comprising (i) a one or more silicone-containing monomers of the general formula: 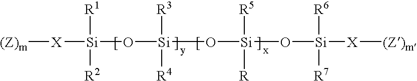
wherein x, y, m, m′, R, R¹, R², R³, R⁴, R⁵, R⁶, R⁷, X, Z and Z′ are as defined herein and (ii) one or more silicon hydride-containing monomers, in a swelling solution comprising one or more solvents and a therapeutically effective amount of at least one pharmaceutically active agent; and (b) removing the polymerization product from the solution to provide the matrix controlled diffusion drug delivery system comprising the therapeutically effective amount of one or more pharmaceutically active agents entrapped in the polymerization product.

In accordance with a fourth embodiment of the present invention, a process for preparing a matrix controlled diffusion drug delivery system sized and configured for back of the eye delivery is provided, the process comprising polymerizing a monomeric mixture comprising (a) one or more silicone-containing monomers of the general formula: 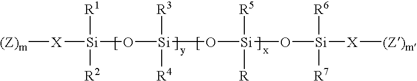
wherein x, y, m, m′, R, R¹, R², R³, R⁴, R⁵, R⁶, R⁷, X, Z and Z′ are as defined herein, and (b) one or more silicon hydride-containing monomers in the presence of a therapeutically effective amount of one or more pharmaceutically active agents to provide the matrix controlled diffusion drug delivery system comprising the therapeutically effective amount of one or more pharmaceutically active agents entrapped in the polymerization product.

In accordance with a fifth embodiment of the present invention, a method for treating a state, disease, disorder, injury or condition in a mammal is provided, the method comprising administering to a mammal in need of such treatment a matrix controlled diffusion drug delivery system comprising a therapeutically effective amount of one or more pharmaceutically active agents entrapped in a polymerization product of a monomeric mixture comprising (a) one or more silicone-containing monomers of the general formula: 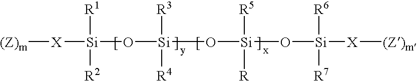
wherein x, y, m, m′, R, R¹, R², R³, R⁴, R⁵, R⁶, R⁷, X, Z and Z′ are as defined herein and (b) one or more silicon hydride-containing monomers, wherein the matrix controlled diffusion drug delivery system is sized and configured for back of the eye delivery.

The drug delivery systems of the present invention may advantageously be designed to allow for manipulation and control of drug release rates, which may be based on the drug to be delivered, the location of delivery, the purpose of delivery and/or the therapeutic requirements of the individual patient such that treatment of a state, disease, disorder, injury or condition in a mammal may be achieved. The matrix controlled diffusion drug delivery system are also advantageously sized and configured for back of the eye delivery of the one or more pharmaceutically active agents.

The term “monomer” and like terms as used herein denote relatively low molecular weight compounds that are polymerizable by, for example, free radical polymerization, as well as higher molecular weight compounds also referred to as “prepolymers”, “macromonomers”, and related terms.

The term “(meth)” as used herein denotes an optional methyl substituent. Accordingly, terms such as “(meth)acrylate” denotes either methacrylate or acrylate, and “(meth)acrylic acid” denotes either methacrylic acid or acrylic acid.

The term “treating” or “treatment” of a state, disease, disorder, injury or condition as used herein shall be understood to mean (1) preventing or delaying the appearance of clinical symptoms of the state, disease, disorder, injury or condition developing in a mammal that may be afflicted with or predisposed to the state, disease, disorder, injury or condition but does not yet experience or display clinical or subclinical symptoms of the state, disease, disorder, injury or condition, (2) inhibiting the state, disease, disorder, injury or condition, i.e., arresting or reducing the development of the disease or at least one clinical or subclinical symptom thereof, or (3) relieving the state, disease, disorder, injury or condition, i.e., causing regression of the state, disease, disorder, injury or condition or at least one of its clinical or subclinical symptoms.

The term “delivering” as used herein shall be understood to mean providing a therapeutically effective amount of a pharmaceutically active agent to a particular location within a host causing a therapeutically effective concentration of the pharmaceutically active agent at the particular location.

The term “subject” or “patient” or “host” or “mammal” as used herein refers to mammalian animals and humans.

### BRIEF DESCRIPTION OF THE DRAWINGS

FIGS. 1-3 are graphical representations depicting the percent cumulative drug release rate over time of timolol maleate from sample implants prepared according to Examples 6-10.

### DETAILED DESCRIPTION OF THE PREFERRED EMBODIMENTS

The present invention relates to matrix controlled diffusion drug delivery systems based on polymerization products derived from a monomeric mixture containing at least one or more silicone-containing monomers and one or more silicon hydride-containing monomers and are sized and configured for back of the eye drug delivery for the treatment of a state, disease, disorder, injury or condition in a mammal need of treatment such as an ophthalmic disease in a mammal. The subject matrix controlled diffusion drug delivery systems advantageously allow for manipulation and control of drug release rates which may be based on, for example, the drug to be delivered, the location of delivery, the purpose of delivery and/or the therapeutic requirements of the individual patient. The rate of release of the pharmaceutically active agents can be controlled by manipulating the hydrophobic/hydrophilic balance of the polymerization product to achieve the desired rate of drug release.

The monomeric mixtures for use in forming matrix controlled diffusion drug delivery systems of the present invention can contain at least (a) one or more silicone-containing monomers and (b) one or more silicon hydride-containing monomers. The silicone-containing monomers of component (a) can be represented by the general formula I: 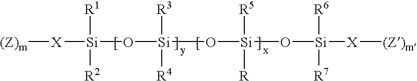
wherein x and y are independently integers from 0 to about 300; m and m′ are independently integers from 1 to about 6, R is independently a straight or branched, substituted or unsubstituted, C₁-C₃₀ alkyl group, a C₁-C₃₀ fluoroalkyl group, an alkyl ether, cycloalkyl ether, cycloalkylalkyl ether, cycloalkenyl ether, aryl ether, arylalkyl ether, a polyether containing group or a C₁-C₃₀ alkylamide group, R¹, R², R³, R⁴, R⁵, R⁶ and R⁷ are independently hydrogen, a straight or branched C₁-C₃₀ alkyl group, a C₁-C₃₀ fluoroalkyl group, a C₁-C₂₀ ester group, an alkyl ether, cycloalkyl ether, cycloalkylalkyl ether, cycloalkenyl ether, aryl ether, arylalkyl ether, a polyether containing group, an ureido group, an amide group, an amine group, a substituted or unsubstituted C₁-C₃₀ alkoxy group, a substituted or unsubstituted C₃-C₃₀ cycloalkyl group, a substituted or unsubstituted C₃-C₃₀ cycloalkylalkyl group, a substituted or unsubstituted C₃-C₃₀ cycloalkenyl group, a substituted or unsubstituted C₅-C₃₀ aryl group, a substituted or unsubstituted C₅-C₃₀ arylalkyl group, a substituted or unsubstituted C₅-C₃₀ heteroaryl group, a substituted or unsubstituted C₃-C₃₀ heterocyclic ring, a substituted or unsubstituted C₄-C₃₀ heterocyclolalkyl group, a substituted or unsubstituted C₆-C₃₀ heteroarylalkyl group, fluorine, a vinyl group, a C₅-C₃₀ fluoroaryl group, or a hydroxyl group; X is independently a straight or branched C₁-C₃₀ alkyl group, a C₁-C₃₀ fluoroalkyl group, a substituted or unsubstituted C₅-C₃₀ arylalkyl group, a substituted or unsubstituted C₁-C₃₀ alkoxy group, an ether, polyether, sulfide, or amino-containing group and Z and Z′ are independently a polymerizable ethylenically unsaturated organic radical.

Representative examples of alkyl groups for use herein include, by way of example, a straight or branched hydrocarbon chain radical containing carbon and hydrogen atoms of from 1 to about 18 carbon atoms with or without unsaturation, to the rest of the molecule, e.g., methyl, ethyl, n-propyl, 1-methylethyl (isopropyl), n-butyl, n-pentyl, etc., and the like.

Representative examples of fluoroalkyl groups for use herein include, by way of example, a straight or branched alkyl group as defined above having one or more fluorine atoms attached to the carbon atom, e.g., —CF₃, —CF₂CF₃, —CH₂CF₃, —CH₂CF₂H, —CF₂H and the like.

Representative examples of ester groups for use herein include, by way of example, a carboxylic acid ester having one to 20 carbon atoms and the like.

Representative examples of ether or polyether containing groups for use herein include, by way of example, an alkyl ether, cycloalkyl ether, cycloalkylalkyl ether, cycloalkenyl ether, aryl ether, arylalkyl ether wherein the alkyl, cycloalkyl, cycloalkylalkyl, cycloalkenyl, aryl, and arylalkyl groups are defined above, e.g., alkylene oxides, poly(alkylene oxide)s such as ethylene oxide, propylene oxide, butylene oxide, poly(ethylene oxide)s, poly(ethylene glycol)s, poly(propylene oxide)s, poly(butylene oxide)s and mixtures or copolymers thereof, an ether or polyether group of the general formula —R⁸OR⁹, wherein R⁸ is a bond, an alkyl, cycloalkyl or aryl group as defined above and R⁹ is an alkyl, cycloalkyl or aryl group as defined above, e.g., —CH₂CH₂OC₆H₅ and —CH₂CH₂OC₂H₅, and the like.

Representative examples of amide groups for use herein include, by way of example, an amide of the general formula —R¹⁰C(O)NR¹¹R¹² wherein R¹⁰, R¹¹ and R¹² are independently C₁-C₃₀ hydrocarbons, e.g., R¹⁰ can be alkylene groups, arylene groups, cycloalkylene groups and R¹¹ and R¹² can be alkyl groups, aryl groups, and cycloalkyl groups as defined herein and the like.

Representative examples of amine groups for use herein include, by way of example, an amine of the general formula —R¹³N R¹⁴R¹⁵ wherein R¹³ is a C₂-C₃₀ alkylene, arylene, or cycloalkylene and R¹⁴ and R¹⁵ are independently C₁-C₃₀ hydrocarbons such as, for example, alkyl groups, aryl groups, or cycloalkyl groups as defined herein, or a quaternary ammonium compound of the general formula 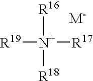
wherein R¹⁶, R¹⁷, R¹⁸ and R¹⁹ are independently C₁-C₃₀ hydrocarbons such as, for example, alkyl groups, aryl groups, or cycloalkyl groups as defined herein and M is an anion, and the like.

Representative examples of an ureido group for use herein include, by way of example, an ureido group having one or more substituents or unsubstituted ureido. The ureido group preferably is an ureido group having 1 to 12 carbon atoms. Examples of the substituents include alkyl groups and aryl groups. Examples of the ureido group include 3-methylureido, 3,3-dimethylureido, and 3-phenylureido.

Representative examples of alkoxy groups for use herein include, by way of example, an alkyl group as defined above attached via oxygen linkage to the rest of the molecule, i.e., of the general formula —OR²⁰, wherein R²⁰ is an alkyl, cycloalkyl, cycloalkylalkyl, cycloalkenyl, aryl or an arylalkyl as defined above, e.g., —OCH₃, —OC₂H₅, or —OC₆H₅, and the like.

Representative examples of cycloalkyl groups for use herein include, by way of example, a substituted or unsubstituted non-aromatic mono or multicyclic ring system of about 3 to about 18 carbon atoms such as, for example, cyclopropyl, cyclobutyl, cyclopentyl, cyclohexyl, perhydronapththyl, adamantyl and norbornyl groups bridged cyclic group or sprirobicyclic groups, e.g., sprio-(4,4)-non-2-yl and the like, optionally containing one or more heteroatoms, e.g., O and N, and the like.

Representative examples of cycloalkylalkyl groups for use herein include, by way of example, a substituted or unsubstituted cyclic ring-containing radical containing from about 3 to about 18 carbon atoms directly attached to the alkyl group which are then attached to the main structure of the monomer at any carbon from the alkyl group that results in the creation of a stable structure such as, for example, cyclopropylmethyl, cyclobutylethyl, cyclopentylethyl and the like, wherein the cyclic ring can optionally contain one or more heteroatoms, e.g., O and N, and the like.

Representative examples of cycloalkenyl groups for use herein include, by way of example, a substituted or unsubstituted cyclic ring-containing radical containing from about 3 to about 18 carbon atoms with at least one carbon-carbon double bond such as, for example, cyclopropenyl, cyclobutenyl, cyclopentenyl and the like, wherein the cyclic ring can optionally contain one or more heteroatoms, e.g., O and N, and the like.

Representative examples of aryl groups for use herein include, by way of example, a substituted or unsubstituted monoaromatic or polyaromatic radical containing from about 5 to about 25 carbon atoms such as, for example, phenyl, naphthyl, tetrahydronapthyl, indenyl, biphenyl and the like, optionally containing one or more heteroatoms, e.g., O and N, and the like.

Representative examples of arylalkyl groups for use herein include, by way of example, a substituted or unsubstituted aryl group as defined above directly bonded to an alkyl group as defined above, e.g., —CH₂C₆H₅, —C₂H₅C₆H₅ and the like, wherein the aryl group can optionally contain one or more heteroatoms, e.g., O and N, and the like.

Representative examples of fluoroaryl groups for use herein include, by way of example, an aryl group as defined above having one or more fluorine atoms attached to the aryl group.

Representative examples of heterocyclic ring groups for use herein include, by way of example, a substituted or unsubstituted stable 3 to about 15 membered ring radical, containing carbon atoms and from one to five heteroatoms, e.g., nitrogen, phosphorus, oxygen, sulfur and mixtures thereof. Suitable heterocyclic ring radicals for use herein may be a monocyclic, bicyclic or tricyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quaternized; and the ring radical may be partially or fully saturated (i.e., heteroaromatic or heteroaryl aromatic). Examples of such heterocyclic ring radicals include, but are not limited to, azetidinyl, acridinyl, benzodioxolyl, benzodioxanyl, benzofurnyl, carbazolyl, cinnolinyl, dioxolanyl, indolizinyl, naphthyridinyl, perhydroazepinyl, phenazinyl, phenothiazinyl, phenoxazinyl, phthalazinyl, pyridyl, pteridinyl, purinyl, quinazolinyl, quinoxalinyl, quinolinyl, isoquinolinyl, tetrazoyl, imidazolyl, tetrahydroisouinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazinyl, pyrimidinyl, pyridazinyl, oxazolyl, oxazolinyl, oxasolidinyl, triazolyl, indanyl, isoxazolyl, isoxasolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzooxazolyl, furyl, tetrahydrofurtyl, tetrahydropyranyl, thienyl, benzothienyl, thiamorpholinyl, thiamorpholinyl sulfoxide, thiamorpholinyl sulfone, dioxaphospholanyl, oxadiazolyl, chromanyl, isochromanyl and the like and mixtures thereof.

Representative examples of heteroaryl groups for use herein include, by way of example, a substituted or unsubstituted heterocyclic ring radical as defined above. The heteroaryl ring radical may be attached to the main structure at any heteroatom or carbon atom that results in the creation of a stable structure.

Representative examples of heteroarylalkyl groups for use herein include, by way of example, a substituted or unsubstituted heteroaryl ring radical as defined above directly bonded to an alkyl group as defined above. The heteroarylalkyl radical may be attached to the main structure at any carbon atom from the alkyl group that results in the creation of a stable structure.

Representative examples of heterocyclo groups for use herein include, by way of example, a substituted or unsubstituted heterocylic ring radical as defined above. The heterocyclo ring radical may be attached to the main structure at any heteroatom or carbon atom that results in the creation of a stable structure.

Representative examples of heterocycloalkyl groups for use herein include, by way of example, a substituted or unsubstituted heterocylic ring radical as defined above directly bonded to an alkyl group as defined above. The heterocycloalkyl radical may be attached to the main structure at carbon atom in the alkyl group that results in the creation of a stable structure.

Representative examples of a “polymerizable ethylenically unsaturated organic radicals” include, by way of example, (meth)acrylate-containing radicals, (meth)acrylamide-containing radicals, vinylcarbonate-containing radicals, vinylcarbamate-containing radicals, styrene-containing radicals and the like. In one embodiment, a polymerizable ethylenically unsaturated organic radical can be represented by the general formula: 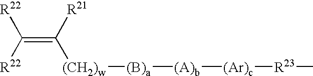
wherein R²¹ is hydrogen or methyl; R²² is independently hydrogen, an alkyl radical having 1 to 6 carbon atoms, or a —CO—Y—R²⁴ radical wherein Y is —O—, —S— or —NH— and R²⁴ is a divalent alkylene radical having 1 to about 10 carbon atoms; R²³ is an alkyl radical having 1 to about 12 carbon atoms; B denotes —CO— or —OCO—; A denotes —O— or —NH—; Ar denotes an aromatic radical having 6 to about 30 carbon atoms; w is 0 to 6; a is 0 or 1; b is 0 or 1; and c is 0 or 1.

The substituents in the ‘substituted alkyl’, ‘substituted alkoxy’, ‘substituted cycloalkyl’, ‘substituted cycloalkylalkyl’, ‘substituted cycloalkenyl’, ‘substituted arylalkyl’, ‘substituted aryl’, ‘substituted heterocyclic ring’, ‘substituted heteroaryl ring’, ‘substituted heteroarylalkyl’, ‘substituted heterocycloalkyl ring’, ‘substituted cyclic ring’ and ‘substituted carboxylic acid derivative’ may be the same or different and include one or more substituents such as hydrogen, hydroxy, halogen, carboxyl, cyano, nitro, oxo (═O), thio(═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, substituted heterocycloalkyl ring, substituted or unsubstituted heteroarylalkyl, substituted or unsubstituted heterocyclic ring, substituted or unsubstituted guanidine, —OORₓ, —C(O)Rₓ, —C(S)Rₓ, —C(O)NRₓR_{y}, —C(O)ONRₓR_{y}, —NRₓCONR_{y}R_{z}, —N(Rₓ)SOR_{y}, —N(Rₓ)SO₂R_{y}, —(═N—N(Rₓ)R_{y}), —NRₓC(O)OR_{y}, —NRₓR_{y}, —NRₓC(O)R_{y}—, —NRₓC(S)R_{y}—NRₓC(S)NR_{y}R_{z}, —SONRₓR_{y}—, —SO₂NRₓR_{y}—, —ORₓ, —ORₓC(O)NR_{y}R_{z}, —ORₓC(O)OR_{y}—, —OC(O)Rₓ, —OC(O)NRₓR_{y}, —RₓNR_{y}C(O)Rₓ, —RₓOR_{y}, —RₓC(O)OR_{y}, —RₓC(O)NR_{y}R_{z}, —RₓC(O)Rₓ, —RₓOC(O)R_{y}, —SRₓ, —SORₓ, —SO₂Rₓ, —ONO₂, wherein Rₓ, R_{y} and R_{z} in each of the above groups can be the same or different and can be a hydrogen atom, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, ‘substituted heterocycloalkyl ring’ substituted or unsubstituted heteroarylalkyl, or a substituted or unsubstituted heterocyclic ring.

In one embodiment, in the silicone-containing monomer x is from 1 to about 300 and preferably from about 20 to about 200, y is from 1 to about 300 and preferably from about 20 to about 200, R is independently a straight or branched C₁-C₃₀ alkyl group, a straight or branched C₁-C₃₀ fluoroalkyl group, an alkyl ether or polyether group and a straight or branched C₁-C₃₀ alkylamide group, R¹, R², R³, R⁴, R⁵, R⁶ and R⁷ are independently a straight or branched, saturated or unsaturated C₁-C₃₀ alkyl group and preferably a C₁-C₆ alkyl group, X is independently a bond or one or more C₁-C₃₀ alkyl ether groups or polyether groups and Z and Z′ are independently vinyl-containing radicals. In one embodiment, a silicon-containing monomer(s) is a vinyl functional siloxane monomer represented by the general formula II: 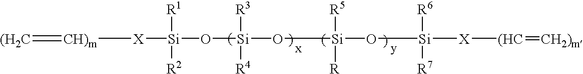
wherein x, y, m, m′, R, R¹, R², R³, R⁴, R⁵, R⁶, R⁷ and X have the aforestated meanings.

Many of the applicable silicon-containing monomers of formulae I and II employed in the monomeric mixtures for use in the drug delivery systems of the present invention are well known in the art and may be synthesized by known techniques and does not constitute a part of the present invention. Numerous examples of such silicon-containing monomers are provided in, e.g., U.S. Pat. Nos. 4,153,641 (Deichert et al.), 5,260,000 (Nandu et al.); 5,310,779 (Lai); 5,387,662 (Kunzler et al.); 5,449,729 (Lai); 5,512,205 (Lai); 5,610,252 (Bambury et al.); 5,616,757 (Bambury et al.); 5,710,302 (Kunzler et al.); 5,714,557 (Kunzler et al.); 5,908,906 (Kunzler et al.); and 6,891,010 (Kunzler et al.), the disclosures of which are incorporated herein by reference. For example, the silicon-containing monomers used herein can be prepared as generally represented in Schemes 1a and 1b below: 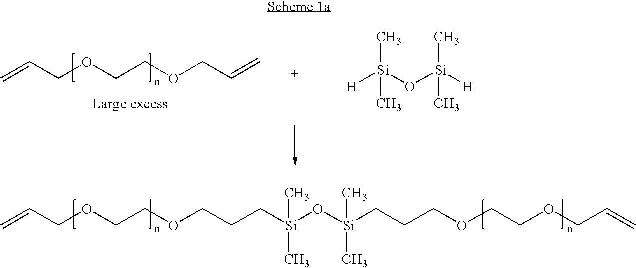
wherein n is 0 to about 10. 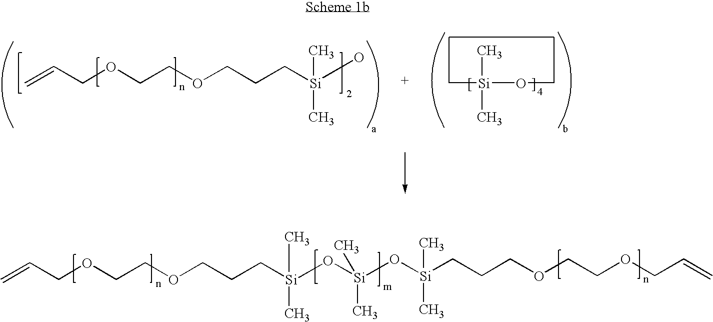
wherein n is 0 to about 10 and m is 0 (when b is 0) to about 100.

The monomeric mixtures herein further contain one or more silicon hydride-containing monomers as crosslinking agents. A wide variety of silicone hydride-containing monomers can be used herein, e.g., hydride functional polysiloxanes, and are commercially available from such sources as Gelest, Inc. (Morrisville, Pa.) and can be prepared by methods well known in the art. In one embodiment, a silicone hydride-containing monomer can be of the general formula III: 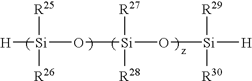
wherein z is from 0 to about 100, R²⁵, R²⁶, R²⁹, and R³⁰ independently are hydrogen, a straight or branched C₁-C₃₀ alkyl group, a C₁-C₃₀ fluoroalkyl group, a C₁-C₂₀ ester group, an ether or polyether containing group, an ureido group, an amide group, an amine group, a substituted or unsubstituted C₁-C₃₀ alkoxy group, a substituted or unsubstituted C₃-C₃₀ cycloalkyl group, a substituted or unsubstituted C₃-C₃₀ cycloalkylalkyl group, a substituted or unsubstituted C₃-C₃₀ cycloalkenyl group, a substituted or unsubstituted C₅-C₃₀ aryl group, a substituted or unsubstituted C₅-C₃₀ arylalkyl group, a substituted or unsubstituted C₅-C₃₀ heteroaryl group, a substituted or unsubstituted C₃-C₃₀ heterocyclic ring, a substituted or unsubstituted C₄-C₃₀ heterocyclolalkyl group, a substituted or unsubstituted C₆-C₃₀ heteroarylalkyl group, fluorine, a vinyl group, a C₅-C₃₀ fluoroaryl group, or a hydroxyl group as defined above, and R²⁷ and R²⁸ independently are a straight or branched C₁-C₃₀ alkyl group, a C₁-C₃₀ fluoroalkyl group, a C₁-C₂₀ ester group, an ether or polyether containing group, an ureido group, an amide group, an amine group, a substituted or unsubstituted C₁-C₃₀ alkoxy group, a substituted or unsubstituted C₃-C₃₀ cycloalkyl group, a substituted or unsubstituted C₃-C₃₀ cycloalkylalkyl group, a substituted or unsubstituted C₃-C₃₀ cycloalkenyl group, a substituted or unsubstituted C₅-C₃₀ aryl group, a substituted or unsubstituted C₅-C₃₀ arylalkyl group, a substituted or unsubstituted C₅-C₃₀ heteroaryl group, a substituted or unsubstituted C₃-C₃₀ heterocyclic ring, a substituted or unsubstituted C₄-C₃₀ heterocyclolalkyl group, a substituted or unsubstituted C₆-C₃₀ heteroarylalkyl group, fluorine, a vinyl group, a C₅-C₃₀ fluoroaryl group, or a hydroxyl group as defined above. Depending on the desired characteristics of the drug delivery device, the amount of crosslinking agent(s) for use herein can be no more than about 20% w/w and preferably no more than about 10% w/w.

Crosslinking of the one or more of the silicone-containing monomers of formula I with the one or more silicon hydride-containing monomers is carried out in the presence of a suitable catalyst, e.g. a platinum disiloxane complex. Suitable catalysts for use herein are well known in the art and are commercially available from such sources as Gelest, Inc. (Morrisville, Pa.). Generally, the amount of catalyst present in the polymerization reaction is an amount sufficient to polymerize the monomeric mixture.

Polymerization of the monomeric mixture can be carried out in any known manner. For example, the component(s) in the reaction mixture can be added continuously to a stirred reactor or can take place in a tubular reactor in which the components can be added at one or more points along the tube. In one embodiment, polymerization may be carried out at a temperature of from about 20° C. to about 100° C. and for a time sufficient to polymerize the silicone-containing monomers(s) and optional monomer(s), e.g., from about 5 minutes to about 16 hours.

In another embodiment, polymerization can be carried out by exposing the reactive monomer(s) and catalyst to, for example, ultraviolet (UV) or visible light or electron beams, in the presence of one or more photoinitiator(s). The use of UV or visible light in combination with photoinitiators is well known in the art and is particularly suitable for formation of the polymerization product. Suitable photoinitiators which are useful for polymerizing the polymerizable mixture of monomers can be commercially available photoinitiators, e.g., photoinitiators commercially available under the “IRGACURE”, “DAROCUR” and “SPEEDCURE” trade names (manufactures by Ciba Specialty Chemicals, also obtainable under a different name from BASF, Fratelli Lamberti and Kawaguchi). They are generally compounds which are capable of initiating the radical reaction of olefinically unsaturated double bonds on exposure to light with a wavelength of, for example, about 260 to about 480 nm.

Generally, pharmaceutically active agents or drugs useful in the matrix controlled diffusion drug delivery systems of the present invention can be any compound, composition of matter, or mixtures thereof that can be delivered from the drug delivery system to produce a beneficial and useful result to, for example, the eye, especially an agent effective in obtaining a desired local or systemic physiological or pharmacological effect. Examples of such agents include, but are not limited to, anesthetics and pain killing agents such as lidocaine and related compounds, benzodiazepam and related compounds and the like; anti-cancer agents such as 5-fluorouracil, adriamycin and related compounds and the like; anti-fungal agents such as fluconazole and related compounds and the like; anti-viral agents such as trisodium phosphomonoformate, trifluorothymidine, acyclovir, ganciclovir, DDI, AZT and the like; cell transport/mobility impending agents such as colchicine, vincristine, cytochalasin B and related compounds and the like; antiglaucoma drugs such as beta-blockers, e.g., timolol, betaxolol, atenalol, and the like; antihypertensives; decongestants such as phenylephrine, naphazoline, tetrahydrazoline and the like; immunological response modifiers such as muramyl dipeptide and related compounds and the like; peptides and proteins such as cyclosporin, insulin, growth hormones, insulin related growth factor, heat shock proteins and related compounds and the like; steroidal compounds such as dexamethasone, prednisolone and related compounds and the like; low solubility steroids such as fluocinolone acetonide and related compounds and the like; carbonic anhydrase inhibitors; diagnostic agents; antiapoptosis agents; gene therapy agents; sequestering agents; reductants such as glutathione and the like; antipermeability agents; antisense compounds; antiproliferative agents; antibody conjugates; antidepressants; bloodflow enhancers; antiasthmatic drugs; antiparasiticagents; non-steroidal anti inflammatory agents such as ibuprofen and the like; nutrients and vitamins: enzyme inhibitors: antioxidants; anticataract drugs; aldose reductase inhibitors; cytoprotectants; cytokines, cytokine inhibitors, and cytokin protectants; uv blockers; mast cell stabilizers; anti neovascular agents such as antiangiogenic agents, e.g., matrix metalloprotease inhibitors and the like.

Representative examples of additional pharmaceutically active agent for use herein include, but are not limited to, neuroprotectants such as nimodipine and related compounds and the like; antibiotics such as tetracycline, chlortetracycline, bacitracin, neomycin, polymyxin, gramicidin, oxytetracycline, chloramphenicol, gentamycin, erythromycin and the like; anti-infectives; antibacterials such as sulfonamides, sulfacetamide, sulfamethizole, sulfisoxazole; nitrofurazone, sodium propionate and the like; antiallergenics such as antazoline, methapyriline, chlorpheniramine; pyrilamine, prophenpyri damine and the like; anti-inflammatories such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triminolone and the like; miotics; anti-cholinesterase such as pilocarpine, eserine salicylate, carbachol, di-isopropyl fluorophosphate, phospholine iodine, demecarium bromide and the like; miotic agents; mydriatics such as atropine sulfate, cyclopentolate, homatropine, scopolamine, tropicamide, eucatropine, hydroxyamphetamine and the like; svmpathomimetics such as epinephrine and the like; and prodrugs such as, for example, those described in Design of Prodrugs, edited by Hans Bundgaard, Elsevier Scientific Publishing Co., Amsterdam, 1985. In addition to the foregoing agents, other agents suitable for treating, managing, or diagnosing conditions in a mammalian organism may be entrapped in the polymerization product and administered using the drug delivery systems of the current invention. Once again, reference may be made to any standard pharmaceutical textbook such as, for example, Remington's Pharmaceutical Sciences for pharmaceutically active agents.

Any pharmaceutically acceptable form of the foregoing pharmaceutically active agent may be employed in the practice of the present invention, e.g., the free base; free acid; pharmaceutically acceptable salts, esters or amides thereof, e.g., acid additions salts such as the hydrochloride, hydrobromide, sulfate, bisulfate, acetate, oxalate, valerate, oleate, palmitate, stearate, laurate, borate, benzoate, lactate, phosphate, tosylate, mesylate, citrate, maleate, fumarate, succinate, tartrate, ascorbate, glucoheptonate, lactobionate, and lauryl sulfate salts and the like; alkali or alkaline earth metal salts such as the sodium, calcium, potassium and magnesium salts and the like; hydrates; enantiomers; isomers; stereoisomers; diastereoisomers; tautomers; polymorphs, mixtures thereof, prodrugs thereof or racemates or racemic mixtures thereof.

Actual dosage levels of the pharmaceutically active agent(s) in the drug delivery systems of the present invention may be varied to obtain an amount of the pharmaceutically active agent(s) that is effective to obtain a desired therapeutic response for a particular system and method of administration. The selected dosage level therefore depends upon such factors as, for example, the desired therapeutic effect, the route of administration, the desired duration of treatment, and other factors. The total daily dose of the pharmaceutically active agent(s) administered to a host in single or divided doses can vary widely depending upon a variety of factors including, for example, the body weight, general health, sex, diet, time and route of administration, rates of absorption and excretion, combination with other drugs, the severity of the particular condition being treated, etc. Generally, the amounts of pharmaceutically active agent(s) present in the drug delivery systems of the present invention can range from about 0.1% w/w to about 60% w/w and preferably from about 1% w/w to about 50% w/w.

Polymerization products of the monomeric mixtures containing at least one or more of the silicon-containing monomers and silicon hydride-containing monomers are combined with one or more pharmaceutically active agents to form the drug delivery systems of the present invention. By controlling the concentration of the hydrophobic and hydrophilic groups on the siloxane backbone, the polar —R group tail, and crosslinking agent, a particular hydrophobic/hydrophilic balance of characteristics or properties may be achieved. The hydrophobic/hydrophilic balance of characteristics may likewise be manipulated to achieve the desired rate of drug release. The desired rate of drug release may be determined based on the drug to be delivered, the location of delivery, the purpose of delivery and/or the therapeutic requirements of the individual patient. The hydrophobic/hydrophilic balance of characteristics dictates the solubility of the drug, and is a primary factor controlling the rate of drug release.

Generally, the drug delivery systems of the present invention can be prepared by first forming the polymerization product of a monomeric mixture as described hereinabove and then entrapping a therapeutically effective amount of one or more of pharmaceutically active agents therein. In one embodiment, the process may include at least (a) forming a polymerization product of a monomeric mixture as described hereinabove; (b) swelling the polymerization product in a swelling solution comprising one or more solvents and a therapeutically effective amount of at least one pharmaceutically active agent; and (c) removing the polymerization product from the solution to provide the matrix controlled diffusion drug delivery system comprising the therapeutically effective amount of one or more pharmaceutically active agents entrapped in the polymerization product.

In step (b) of the process, the polymerization product can be swelled with the swelling solution by, for example, immersing the polymerization product in the swelling solution for a time period sufficient to entrap the pharmaceutically active agent in the polymerization product, e.g., a time period of from about 1 hour to about 24 hours. The swelling solution will ordinarily include at least one or more solvents and a therapeutically effective amount of the one or more pharmaceutically active agents. Suitable solvents include, but are not limited to, ketones, alcohols, ethers, aliphatic hydrocarbons, aromatic hydrocarbons, sulfoxides, amide-based solvents and the like and mixtures thereof.

Ketones for use herein can be one or more ketones of the general formula R³⁰R³¹C(O) wherein R³¹ and R³² are the same or different and can be a substituted or unsubstituted C₁-C₃₀ alkyl, a substituted or unsubstituted C₃-C₃₀ cycloalkyl, a substituted or unsubstituted C₃-C₃₀ cycloalkylalkyl, a substituted or unsubstituted C₃-C₃₀ cycloalkenyl, a substituted or unsubstituted C₅-C₃₀ aryl, a substituted or unsubstituted a C₅-C₃₀ arylalkyl, a substituted or unsubstituted C₅-C₃₀ heteroaryl, a substituted or unsubstituted C₃-C₃₀ heterocyclic ring, a substituted or unsubstituted C₄-C₃₀ heterocyclylalkyl, a substituted or unsubstituted C₆-C₃₀ heteroarylalkyl; or R³¹ and R³² together with the carbon atom to which they are bonded are joined together to form a saturated or unsaturated ring optionally containing one or more heterocyclic atoms. In another embodiment, the ketones for use herein include those containing at least three carbon atoms. Representative examples of ketones for use herein include, but are not limited to, acetone, methyl ethyl ketone, diethyl ketone, methyl propyl ketone, methyl isopropyl ketone, ethyl propyl ketone, ethyl isopropyl ketone, dipropyl ketone, diisopropyl ketone, methyl butyl ketone, methyl isobutyl ketone, methyl sec butyl ketone, methyl tert-butyl ketone, ethyl butyl ketone, ethyl isobutyl ketone, ethyl sec-butyl ketone, ethyl tert-butyl ketone, propyl butyl ketone, isopropyl butyl ketone, propyl isobutyl ketone, propyl sec-butyl ketone, propyl tert butyl ketone, isopropyl isobutyl ketone, isopropyl sec-butyl ketone, isopropyl tert-butyl ketone, dibutyl ketone, diisobutyl ketone, di-sec-butyl ketone, di-tert-butyl ketone, butyl isobutyl ketone, butyl sec-butyl ketone, butyl tert-butyl ketone, isobutyl sec-butyl ketone, isobutyl tert-butyl ketone, sec-butyl tert-butyl ketone, 5-heptanone, 5-methyl-2-hexanone (methyl isoamyl ketone), 4-methyl-2-hexanone, 3-methyl-2-hexanone, 3,4-dimethyl-2-pentanone, 3,3-dimethyl-2-pentanone, 4,4-dimethyl-2-pentanone, 3-octanone, 4-methyl-3-heptanone, 5-methyl-3-heptanone, 6-methyl-3-heptanone, 4,4-dimethyl-3-hexanone, 4,5-dimethyl-3-hexanone, 5,5-dimethyl-3-hexanone, 4-nonanone, 5-methyl-4-octanone, 6-methyl-4-octanone, 7-methyl-4-octanone, 5,5-dimethyl-4-neptanone, 5,6-dimethyl-4-heptanone, 6,6-dimethyl-4-heptanone, 2-undecanone, cyclopropanone, cyclobutanone, cyclopentanone, cyclohexanone, cycloheptanone, cyclooctanone, cyclononanone, cyclodecanone, cycloundecanone, cyclododecanone and the like and combinations thereof. A preferred ketone for use herein is acetone.

Alcohols for use herein include, but are not limited to, C₁-C₃₀ aliphatic alcohols, C₆-C₃₀ aromatic alcohols and the like and mixtures thereof. Examples of useful alcohols include, but are not limited to, methyl alcohol, ethyl alcohol, propyl alcohol, isopropyl alcohol, butyl alcohol, benzyl alcohol and the like and mixtures thereof. A preferred alcohol for use herein is isopropyl alcohol.

Suitable ether solvents for use herein include, but are not limited to, tetrahydrofuran and the like and mixtures thereof. Suitable aliphatic hydrocarbon solvents for use herein include, but are not limited to, hexane, heptane, and the like and mixtures thereof. Suitable aromatic hydrocarbon solvents for use herein include, but are not limited to, toluene, benzene, xylene and the like and mixtures thereof. Suitable sulfoxide solvents for use herein include, but are not limited to, dimethylsulfoxide (DMSO), sulfolane and the like and mixtures thereof. Suitable amide-based solvents for use herein include, but are not limited to, pyrrolidine, N-methylpyrrolidine, N,N-dimethylformamide (DMF), N,N-dimethylacetamide (DMA) and the like and mixtures thereof.

Generally, the amount of solvent employed in the solution will range from about 5% w/w to about 500% w/w and preferably from about 10% w/w to about 200% w/w.

Next, the polymerization product will be taken out of the solution and the solvent will be substantially removed from the swelled polymerization product to provide a drug delivery system of the present invention. The solvent can be substantially removed from the swelled polymerization product by techniques known in the art such as, for example, drying, e.g., air drying, vacuum drying, freeze drying, drying under an inert gas (e.g., nitrogen), in an oven at elevated temperatures and the like.

In an alternate embodiment, the drug delivery systems of the present invention can be prepared by polymerizing the monomeric mixture under polymerization conditions as discussed above in the presence of one or more pharmaceutically active agents such that the pharmaceutically active agent(s) is entrapped in the polymerization product. As one skilled in the art will readily appreciate, the resulting polymerization product may have some pharmaceutically active agent(s) which is covalently bound to the polymerization product as well as some free starting monomer(s). If desired, these reactants may be removed from the resulting product by conventional techniques.

The matrix controlled diffusion drug delivery systems of the present invention may be manufactured in any suitable form, shape, e.g., circular, rectangular, tubular, square and triangular shapes, or size suitable for the treatment which they are intended to be used. Methods of forming the subject matrix controlled diffusion drug delivery systems include, but are not limited to, cast molding, injection/compression molding, extrusion, and other methods known to those skilled in the art. For example, for use as an inner back of the eye implant, the drug delivery system may be a hollow cylinder or tube having a first cross dimension (diameter, width) ranging from about 0.025 mm to about 10 mm and a second cross dimension, such as length, from about 0.2 mm to about 10 mm.

Alternatively, the drug delivery system can be in the form of a solution, suspension, solution/suspension, microsphere or nanosphere using a pharmaceutically acceptable carrier well known in the art. Additionally, the solution, suspension, solution/suspension, microsphere or nanosphere can contain one or more pharmaceutically acceptable excipients such as suspending agents, e.g., sodium carboxymethyl cellulose, methylcellulose, hydroxypropylmethylcellulose, sodium alginate, poly(N-vinylpyrrolidone), gum tragacanth and gum acacia; dispersing or wetting agents, e.g., naturally occurring phosphatide, e.g., lecithin, or condensation products of an alkylene oxide with fatty acids, e.g., polyoxyethylene stearate, or condensation products of ethylene oxide with long chain aliphatic alcohols, e.g., heptadecaethylene-oxycetanol, or condensation products of ethylene oxide with partial esters derived from fatty acids and a hexitol, e.g., polyoxyethylene sorbitol monoleate or condensation products of ethylene oxide with partial esters derived from fatty acids and hexitol anhydrides, e.g., polyoxyethylene sorbitan monoleate. Once manufactured, the subject matrix controlled diffusion drug delivery systems are packaged and sterilized using customary methods known to those skilled in the art.

Matrix controlled diffusion drug delivery systems of the present invention may be used in a broad range of therapeutic applications. The matrix controlled diffusion drug delivery systems of the present invention are particularly useful in the treatment of ophthalmic diseases, disorders and/or injuries. Representative examples of such ophthalmic diseases, disorders or injuries include, but are not limited to, diabetic retinopathy, glaucoma, macular degeneration, retinitis pigmentosa, retinal tears or holes, retinal-detachment, retinal ischemia, acute retinopathies associated with trauma, inflammatory mediated degeneration, post-surgical complications, damage associated with laser therapy including photodynamic therapy (PDT), surgical light induced iatrogenic retinopathy, drug-induced retinopathies, autosomal dominant optic atrophy, toxic/nutritional amblyopias; leber's hereditary optic neuropathy (LHOP), other mitochondrial diseases with ophthalmic manifestations or complications, angiogenesis; atypical RP; bardet-biedl syndrome; blue-cone monochromacy; cataracts; central areolar choroidal dystrophy; choroideremia; cone dystrophy; rod dystrophy; cone-rod dystrophy; rod-cone dystrophy; congenital stationary night blindness; cytomegalovirus retinitis; diabetic macular edema; dominant drusen; giant cell arteritis (GCA); goldmann-favre dystrophy; graves' ophthalmopathy; gyrate atrophy; hydroxychloroquine; iritis; juvenile retinoschisis; kearns-sayre syndrome; lawrence-moon bardet-biedl syndrome; leber congenital amaurosis; lupus-induced cotton wool spots; macular degeneration, dry form; macular degeneration, wet form; macular drusen; macular dystrophy; malattia leventinese; ocular histoplasmosis syndrome; oguchi disease; oxidative damage; proliferative vitreoretinopathy; refsum disease; retinitis punctata albescens; retinopathy of prematurity; rod monochromatism; RP and usher syndrome; scleritis; sector RP; sjogren-larsson syndrome; sorsby findus dystrophy; stargardt disease and other retinal diseases.

The drug delivery systems of the present invention can be administered to a mammal in need of treatment by way of a variety of routes. For example, the drug delivery systems may be used by implantation within a portion of the body in need of localized drug delivery, e.g., the interior portion of an eye. However, the subject matrix controlled diffusion drug delivery systems may likewise be used in accordance with other surgical procedures known to those skilled in the field of ophthalmology. For example, the drug delivery systems can be administered to the region of the eye in need of treatment employing instruments known in the art, e.g., a flexible microcatheter system or cannula disclosed in U.S. Patent Application Publication No. 2002/0002362, or the intraretinal delivery and withdrawal systems disclosed in U.S. Pat. Nos. 5,273,530 and 5,409,457, the contents of each of which are incorporated by reference herein. The pharmaceutically active agent may be released from the drug delivery device over a sustained and extended period of time. Optionally, the drug release rate may also be controlled through the attachment of an inert diffusion barrier by way of, for example, surface treatment of the drug delivery device. The surface treatment may be applied through a variety of surface treatment techniques known in the art, e.g., oxidative plasma, evaporative deposition, dip coating or extrusion techniques.

The following examples are provided to enable one skilled in the art to practice the invention and are merely illustrative of the invention. The examples should not be read as limiting the scope of the invention as defined in the claims.

### EXAMPLE 1

### Synthesis of Diallyl Triethylene Glycol Terminated Disiloxane

To a 1 L round bottom flask equipped with a magnetic stirrer and water condenser is added tetramethyldisiloxane (10 grams, 0.074 moles), diallyl terminated diethylene glycol (69 grams, 0.37 moles), tetramethyldisiloxane platinum complex available from Gelest, Inc. (Morrisville, Pa.) (40 mL), dioxane (200 mL) and anhydrous tetrahydrofuran (200 mL) under a nitrogen blanket. The reaction mixture is heated to 75° C. and the reaction was monitored by IR and ¹H-NMR spectroscopy for loss of silicone hydride. The reaction is completed in 4 to 5 hours of reflux. The resulting solution is placed on a rotoevaporator to remove tetrahydrofuran and dioxane. The resultant crude product is passed through a column of silica gel using standard chromatography techniques. The collected solution is again placed on the rotoevaporator to remove solvent and the resultant clear fluid is placed under vacuum (>0.1 mm Hg) at 50° C. for four hours. The resulting diallyl terminated disiloxane is a clear fluid.

### EXAMPLE 2

### Synthesis of a Diallyl Terminated Siloxane

To a 500 mL round bottom flask under dry nitrogen is added D₄ (octamethylcyclotetrasiloxane) (93.59 grams, 0.316 moles) and the disiloxane (6.36 grams, 0.0126 moles) prepared in Example 1. Trifluoromethane sulfonic acid (0.25% w/w) is added as initiator. The reaction mixture is stirred 24 hours with vigorous stirring at room temperature. Sodium bicarbonate (10 fold excess based on trifluoromethane-sulfonic acid) is then added and the reaction mixture is again stirred for 24 hours. The resultant solution is filtered through a 0.3% Teflon® filter. (E.I. DuPont De Nemours & Co., Wilmington, Del.). The filtered solution is vacuum stripped and placed under vacuum (>0.1 mm Hg) at 50° C. to remove the unreacted silicone cyclics. The resulting diallyl terminated siloxane is believed to be a viscous, clear fluid.

### EXAMPLE 3

### Synthesis of Silicone Hydride

To a 1000 mL round bottom flask under dry nitrogen is added D₄ (77.14 grams, 0.26 moles), D₄H (tetramethylcyclotetrasiloxane) (20.85 grams, 0.087 moles) and hexamethyldisiloxane (1.86 grams, 0.0138 moles). Trifluoromethane-sulfonic acid (0.25% w/w) is added as initiator. The reaction mixture is stirred 24 hours with vigorous stirring at room temperature. Sodium bicarbonate (10 fold excess based on trifluoromethane-sulfonic acid) is then added and the reaction mixture is again stirred for 24 hours. The resultant solution is filtered through a 0.3μ Teflon® filter (E.I. DuPont De Nemours & Co., Wilmington, Del.). The filtered solution is vacuum stripped and placed under vacuum (>0.1 mm Hg) at 50° C. to remove the unreacted silicone cyclics. The resulting silicone hydride functionalized siloxane is a viscous, clear fluid.

### EXAMPLE 4

To 90 parts of the diallyl terminated siloxane prepared in example 2 is added 10 parts of the hydride containing siloxane prepared in Example 3 and a catalytic amount of a platinum disiloxane complex. To this reaction mixture is added 20% w/w of Timolol Maleate (TM). The suspension is added to Teflon® tubes available from Boramed, Inc. (Durham, N.C.) (0.5 mm in diameter) and polymerized at 60° C. for four hours. Following the cure, the drug loaded copolymer is removed from the tube resulting in a release device having dimensions of 5 mm by 0.5 mm.

### EXAMPLE 5

### Drug Release

The sample as prepared in Example 4 is placed in 3 cc of borate buffer in a sealed glass tube and the amount of TM release is monitored at 34° C. At periodic intervals, 3 cc of solution is removed and replaced with 3 cc of fresh borate. The solution is analyzed by liquid chromatography for TM. The release rate per day and percent cumulative release were determined. A zero-order drug release is obtained shortly after the initial burst.

### EXAMPLE 6

Implant Preparation

To Med 6-6812 Part A (1.0164 g) (a vinyl functional siloxane obtained from NuSil Technologies, LLC., Carpinteria, Calif.) and Med 6-6812 Part B (0.1052 g.) (a silicon hydride prepolymer obtained from NuSil Technologies, LLC., Carpinteria, Calif.) was added Timolol maleate (0.1014 g.) (commercially available) with mixing.

The mixture was injected into a 0.022” ID FEP fluoropolymer tubing available from Boramed, Inc. (Durham, N.C.) using a syringe with a 23 gauge needle and polymerized at 65° C. for fifteen hours. Following the cure, the drug loaded copolymer was removed from the tube and cut into approximately 7 mm. lengths. The implant formed comprised 8.30 wt. % timolol maleate (6.07 wt. % timolol; 2.23 wt. % maleate).

Testing

Initial release testing was conducted by placing 2 implants prepared according to the formulation above into three separate vials along with 3 mls of Phosphate buffered saline (PBS).

A (2.59 mg=214.7 mg Timolol Maleate=157.1 mg Timolol

B (2.59 mg=214.7 mg Timolol Maleate=157.1 mg Timolol

C (2.38 mg=197.5 mg Timolol Maleate=144.5 mg Timolol

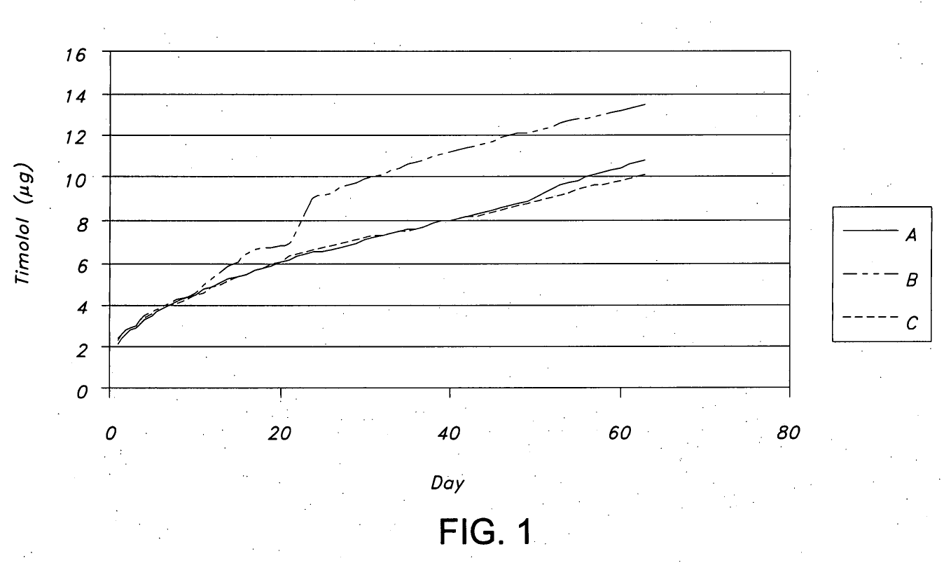

The vials were then placed on a Titer Plate Shaker (Model 4625) and shaken at 160 rotations per minute to monitor the amount of TM release at 37° C. At periodic intervals, 3 mls of PBS was removed and replaced with 3 mls of fresh PBS. The solution was analyzed by high performance liquid chromatography (HPLC) to determine the Timolol concentration. The cumulative release was determined as illustrated in FIG. 1.

### EXAMPLES 7-10

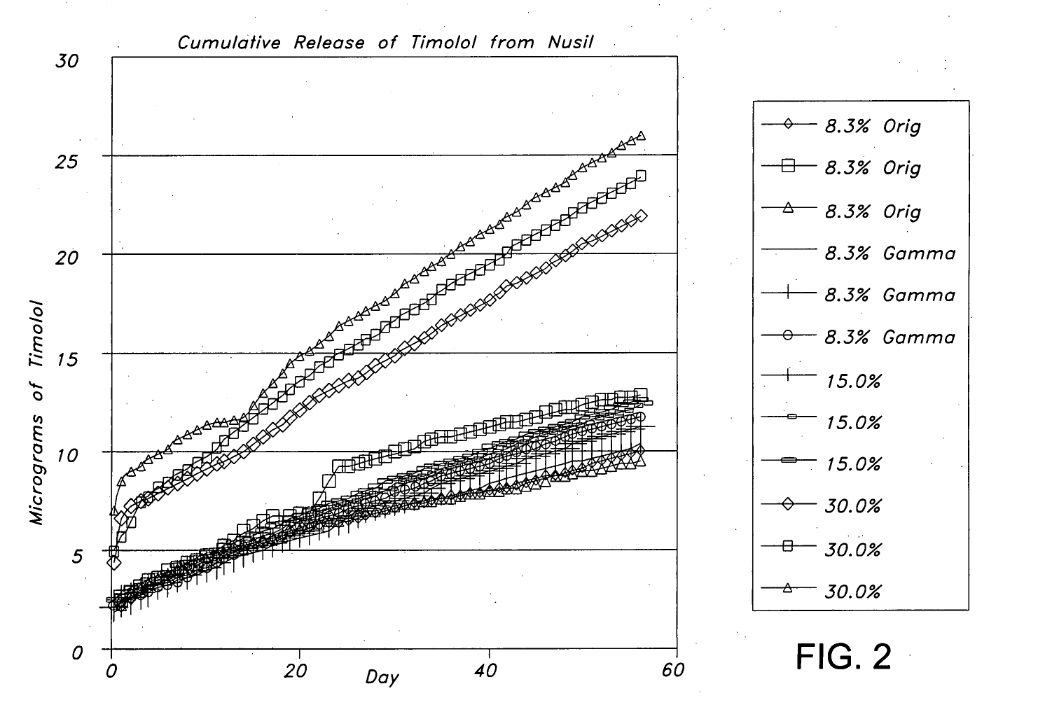

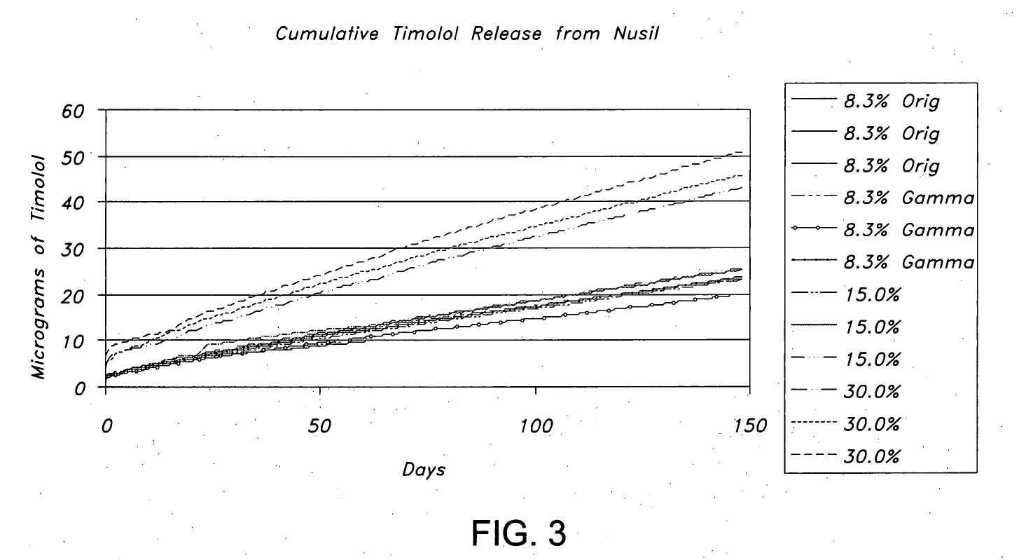

To test the effect of varying timolol maleate concentration on the release profile, implants were prepared substantially in the same manner as in Example 6. The implants were prepared with varying concentrations of timolol maleate to provide implants having concentrations of 8.3 wt % timolol maleate (Example 7), 8.3 wt % gamma radiated timolol maleate (Example 8), 15 wt % timolol maleate (Example 9) and 30 wt % timolol maleate (Example 10). The 8.3 wt % timolol maleate implants were subjected to standard gamma sterilization (Example 10) to determine if exposure to gamma irradiation would have an impact on the release profile. Each of the implants was tested in substantially the same manner as in Example 6. The results of this testing can be found in FIGS. 2 and 3.

The results shown in FIGS. 2 and 3 demonstrate that gamma sterilization does not negatively affect the release profile of the implants prepared according to the formulation provided above. Although not wishing to be bound by a particular theory, the inventors believe that the reason that the 8.3 wt. % implants and the 15 wt. % implants have substantially the same release profile is because they were prepared on different days and therefore the timolol maleate contained in the implants formed may not have had the same particle size. It is believed that the particle size of the timolol maleate in the formulations is determined by the mixing conditions as the timolol maleate used to make the formulations comprised a variety of particle sizes.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. A matrix controlled diffusion drug delivery system comprising a therapeutically effective amount of one or more pharmaceutically active agents entrapped in a polymerization product of a monomeric mixture comprising (a) one or more silicone-containing monomers of the general formula: 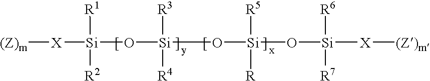
wherein x and y are independently integers from 0 to about 300; m and m′ are independently integers from 1 to about 6, R is independently a straight or branched, substituted or unsubstituted, C₁-C₃₀ alkyl group, a C₁-C₃₀ fluoroalkyl group, an alkyl ether, cycloalkyl ether, cycloalkylalkyl ether, cycloalkenyl ether, aryl ether, arylalkyl ether or polyether containing group or a C₁-C₃₀ alkylamide group, R¹, R², R³, R⁴, R⁵, R⁶ and R⁷ are independently hydrogen, a straight or branched C₁-C₃₀ alkyl group, a C₁-C₃₀ fluoroalkyl group, a C₁-C₂₀ ester group, an alkyl ether, cycloalkyl ether, cycloalkylalkyl ether, cycloalkenyl ether, aryl ether, arylalkyl ether or polyether containing group, an ureido group, an amide group, an amine group, a substituted or unsubstituted C₁-C₃₀ alkoxy group, a substituted or unsubstituted C₃-C₃₀ cycloalkyl group, a substituted or unsubstituted C₃-C₃₀ cycloalkylalkyl group, a substituted or unsubstituted C₃-C₃₀ cycloalkenyl group, a substituted or unsubstituted C₅-C₃₀ aryl group, a substituted or unsubstituted C₅-C₃₀ arylalkyl group, a substituted or unsubstituted C₅-C₃₀ heteroaryl group, a substituted or unsubstituted C₃-C₃₀ heterocyclic ring, a substituted or unsubstituted C₄-C₃₀ heterocyclolalkyl group, a substituted or unsubstituted C₆-C₃₀ heteroarylalkyl group, fluorine, a vinyl group, a C₅-C₃₀ fluoroaryl group, or a hydroxyl group; X is independently a bond, a straight or branched C₁-C₃₀ alkyl group, a C₁-C₃₀ fluoroalkyl group, a substituted or unsubstituted C₅-C₃₀ arylalkyl group, a substituted or unsubstituted C₁-C₃₀ alkoxy group, an ether, polyether, sulfide, or amino-containing group and Z and Z′ are independently a polymerizable ethylenically unsaturated organic radical, and (b) one or more silicon hydride-containing monomers, wherein the matrix controlled diffusion drug delivery system is sized and configured for back of the eye delivery.

2. The matrix controlled diffusion drug delivery system of claim 1, wherein x is 2 to about 100, y is 2 to about 100, R¹, R², R³, R⁴, R⁵, R⁶ and R⁷ are independently a straight or branched C₁-C₃₀ alkyl group, and Z and Z′ independently are vinyl-containing radicals.

3. The matrix controlled diffusion drug delivery system of claim 1, wherein x is 2 to about 100, y is 2 to about 100, R¹, R², R³, R⁴, R⁵, R⁶ and R⁷ are independently a straight or branched C₁-C₆ alkyl group X is a bond or an ether or polyether containing group, and Z and Z′ independently are vinyl-containing radicals.

4. The matrix controlled diffusion drug delivery system of claim 1, wherein x is 2 to about 100, y is 2 to about 100, R is independently a straight or branched C₁-C₃₀ fluoroalkyl group, an alkyl ether or polyether group or a straight or branched C₁-C₃₀ alkylamide group, R¹, R², R³, R⁴, R⁵, R⁶ and R⁷ are independently a straight or branched C₁-C₆ alkyl group, X is independently a bond or one or more C₁-C₃₀ alkyl ether groups or polyether groups and Z and Z′ independently are vinyl-containing radicals.

5. The matrix controlled diffusion drug delivery system of claim 1, wherein the one or more pharmaceutically active agents is selected from the group consisting of an anti-glaucoma agent, anti-cataract agent, anti-diabetic retinopathy agent, thiol cross-linking agent, anti-cancer agent, immune modulator agent, anti-clotting agent, anti-tissue damage agent, anti-inflammatory agent, anti-fibrous agent, non-steroidal anti-inflammatory agent, antibiotic, anti-pathogen agent, piperazine derivative, cycloplegic agent, miotic agent, mydriatic agent and mixtures thereof.

6. The matrix controlled diffusion drug delivery system of claim 1, wherein the one or more pharmaceutically active agents is selected from the group consisting of an anticholinergic, anticoagulant, antifibrinolytic, antihistamine, antimalarial, antitoxin, chelating agent, hormone, immunosuppressive, thrombolytic, vitamin, protein, salt, desensitizer, prostaglandin, amino acid, metabolite, antiallergenic and mixtures thereof.

7. The matrix controlled diffusion drug delivery system of claim 1, wherein the silicon hydride-containing monomer is of the general formula: 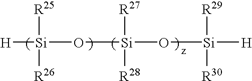
wherein z is from 0 to about 100, R²⁵, R²⁶, R²⁹, and R³⁰ independently are hydrogen, a straight or branched C₁-C₃₀ alkyl group, a C₁-C₃₀ fluoroalkyl group, a C₁-C₂₀ ester group, an ether or polyether containing group, an ureido group, an amide group, an amine group, a substituted or unsubstituted C₁-C₃₀ alkoxy group, a substituted or unsubstituted C₃-C₃₀ cycloalkyl group, a substituted or unsubstituted C₃-C₃₀ cycloalkylalkyl group, a substituted or unsubstituted C₃-C₃₀ cycloalkenyl group, a substituted or unsubstituted C₅-C₃₀ aryl group, a substituted or unsubstituted C₅-C₃₀ arylalkyl group, a substituted or unsubstituted C₅-C₃₀ heteroaryl group, a substituted or unsubstituted C₃-C₃₀ heterocyclic ring, a substituted or unsubstituted C₄-C₃₀ heterocyclolalkyl group, a substituted or unsubstituted C₆-C₃₀ heteroarylalkyl group, fluorine, a vinyl group, a C₅-C₃₀ fluoroaryl group, or a hydroxyl group, and R²⁷ and R²⁸ independently are a straight or branched C₁-C₃₀ alkyl group, a C₁-C₃₀ fluoroalkyl group, a C₁-C₂₀ ester group, an ether or polyether containing group, an ureido group, an amide group, an amine group, a substituted or unsubstituted C₁-C₃₀ alkoxy group, a substituted or unsubstituted C₃-C₃₀ cycloalkyl group, a substituted or unsubstituted C₃-C₃₀ cycloalkylalkyl group, a substituted or unsubstituted C₃-C₃₀ cycloalkenyl group, a substituted or unsubstituted C₅-C₃₀ aryl group, a substituted or unsubstituted C₅-C₃₀ arylalkyl group, a substituted or unsubstituted C₅-C₃₀ heteroaryl group, a substituted or unsubstituted C₃-C₃₀ heterocyclic ring, a substituted or unsubstituted C₄-C₃₀ heterocyclolalkyl group, a substituted or unsubstituted C₆-C₃₀ heteroarylalkyl group, fluorine, a vinyl group, a C₅-C₃₀ fluoroaryl group, or a hydroxyl group.

8. The matrix controlled diffusion drug delivery system of claim 1, in a form of a solution, suspension, solution/suspension, microsphere or nanosphere.

9. The matrix controlled diffusion drug delivery system of claim 1, in a form of a semi-solid or solid article suitable for ocular implant.

10. A process for preparing a matrix controlled diffusion drug delivery system sized and configured for back of the eye delivery, the process comprising entrapping a therapeutically effective amount of one or more pharmaceutically active agents in a polymerization product of a monomeric mixture comprising (a) one or more silicone-containing monomers of the general formula: 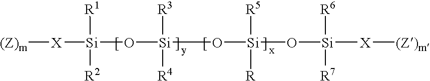
wherein x and y are independently integers from 0 to about 300; m and m′ are independently integers from 1 to about 6, R is independently a straight or branched, substituted or unsubstituted, C₁-C₃₀ alkyl group, a C₁-C₃₀ fluoroalkyl group, an alkyl ether, cycloalkyl ether, cycloalkylalkyl ether, cycloalkenyl ether, aryl ether, arylalkyl ether or polyether containing group or a C₁-C₃₀ alkylamide group, R¹, R², R³, R⁴, R⁵, R⁶ and R⁷ are independently hydrogen, a straight or branched C₁-C₃₀ alkyl group, a C₁-C₃₀ fluoroalkyl group, a C₁-C₂₀ ester group, an alkyl ether, cycloalkyl ether, cycloalkylalkyl ether, cycloalkenyl ether, aryl ether, arylalkyl ether or polyether containing group, an ureido group, an amide group, an amine group, a substituted or unsubstituted C₁-C₃₀ alkoxy group, a substituted or unsubstituted C₃-C₃₀ cycloalkyl group, a substituted or unsubstituted C₃-C₃₀ cycloalkylalkyl group, a substituted or unsubstituted C₃-C₃₀ cycloalkenyl group, a substituted or unsubstituted C₅-C₃₀ aryl group, a substituted or unsubstituted C₅-C₃₀ arylalkyl group, a substituted or unsubstituted C₅-C₃₀ heteroaryl group, a substituted or unsubstituted C₃-C₃₀ heterocyclic ring, a substituted or unsubstituted C₄-C₃₀ heterocyclolalkyl group, a substituted or unsubstituted C₆-C₃₀ heteroarylalkyl group, fluorine, a vinyl group, a C₅-C₃₀ fluoroaryl group, or a hydroxyl group; X is independently a bond, a straight or branched C₁-C₃₀ alkyl group, a C₁-C₃₀ fluoroalkyl group, a substituted or unsubstituted C₅-C₃₀ arylalkyl group, a substituted or unsubstituted C₁-C₃₀ alkoxy group, an ether, polyether, sulfide, or amino-containing group and Z and Z′ are independently a polymerizable ethylenically unsaturated organic radical, and (b) one or more silicon hydride-containing monomers.

11. The process of claim 10, wherein the step of entrapping the therapeutically effective amount of one or more pharmaceutically active agents in the polymerization product comprises polymerizing the monomeric mixture in the presence of the therapeutically effective amount of one or more pharmaceutically active agents.

12. A process for preparing a matrix controlled diffusion drug delivery system, the process comprising (a) swelling a polymerization product of a monomeric mixture comprising (i) one or more silicone-containing monomers of the general formula: 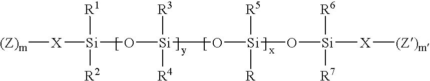
wherein x and y are independently integers from 0 to about 300; m and m′ are independently integers from 1 to about 6, R is independently a straight or branched, substituted or unsubstituted, C₁-C₃₀ alkyl group, a C₁-C₃₀ fluoroalkyl group, an alkyl ether, cycloalkyl ether, cycloalkylalkyl ether, cycloalkenyl ether, aryl ether, arylalkyl ether or polyether containing group or a C₁-C₃₀ alkylamide group, R¹, R², R³, R⁴, R⁵, R⁶ and R⁷ are independently hydrogen, a straight or branched C₁-C₃₀ alkyl group, a C₁-C₃₀ fluoroalkyl group, a C₁-C₂₀ ester group, an alkyl ether, cycloalkyl ether, cycloalkylalkyl ether, cycloalkenyl ether, aryl ether, arylalkyl ether or polyether containing group, an ureido group, an amide group, an amine group, a substituted or unsubstituted C₁-C₃₀ alkoxy group, a substituted or unsubstituted C₃-C₃₀ cycloalkyl group, a substituted or unsubstituted C₃-C₃₀ cycloalkylalkyl group, a substituted or unsubstituted C₃-C₃₀ cycloalkenyl group, a substituted or unsubstituted C₅-C₃₀ aryl group, a substituted or unsubstituted C₅-C₃₀ arylalkyl group, a substituted or unsubstituted C₅-C₃₀ heteroaryl group, a substituted or unsubstituted C₃-C₃₀ heterocyclic ring, a substituted or unsubstituted C₄-C₃₀ heterocyclolalkyl group, a substituted or unsubstituted C₆-C₃₀ heteroarylalkyl group, fluorine, a vinyl group, a C₅-C₃₀ fluoroaryl group, or a hydroxyl group; X is independently a bond, a straight or branched C₁-C₃₀ alkyl group, a C₁-C₃₀ fluoroalkyl group, a substituted or unsubstituted C₅-C₃₀ arylalkyl group, a substituted or unsubstituted C₁-C₃₀ alkoxy group, an ether, polyether, sulfide, or amino-containing group and Z and Z′ are independently a polymerizable ethylenically unsaturated organic radical, and (ii) one or more silicon hydride-containing monomers, in a swelling solution comprising one or more solvents and a therapeutically effective amount of one or more pharmaceutically active agents; and (b) removing the polymerization product from the solution to provide the matrix controlled diffusion drug delivery system comprising the therapeutically effective amount of one or more pharmaceutically active agents entrapped in the polymerization product.

13. The process of claim 12, wherein x is 2 to about 100, y is 2 to about 100, R¹, R², R³, R⁴, R⁵, R⁶ and R⁷ are independently a straight or branched C₁-C₃₀ alkyl group, and Z and Z′ are independently vinyl-containing radicals.

14. The process of claim 12, wherein x is 2 to about 100, y is 2 to about 100, R¹, R², R³, R⁴, R⁵, R⁶ and R⁷ are independently a straight or branched C₁-C₆ alkyl group X is a bond or an ether or polyether containing group, and Z and Z′ are independently vinyl-containing radicals.

15. The process of claim 12, wherein x is 2 to about 100, y is 2 to about 100, R is independently a straight or branched C₁-C₃₀ fluoroalkyl group, an alkyl ether or polyether group or a straight or branched C₁-C₃₀ alkylamide group, R¹, R², R³, R⁴, R⁵, R⁶ and R⁷ are independently a straight or branched C₁-C₆ alkyl group, X is independently a bond or one or more C₁-C₃₀ alkyl ether groups or polyether groups and Z and Z′ are independently vinyl-containing radicals.

16. The process of claim 12, wherein the one or more pharmaceutically active agents is selected from the group consisting of an anti-glaucoma agent, anti-cataract agent, anti-diabetic retinopathy agent, thiol cross-linking agent, anti-cancer agent, immune modulator agent, anti-clotting agent, anti-tissue damage agent, anti-inflammatory agent, anti-fibrous agent, non-steroidal anti-inflammatory agent, antibiotic, anti-pathogen agent, piperazine derivative, cycloplegic agent, miotic agent, mydriatic agent and mixtures thereof.

17. The process of claim 12, wherein the one or more pharmaceutically active agents is selected from the group consisting of an anticholinergic, anticoagulant, antifibrinolytic, antihistamine, antimalarial, antitoxin, chelating agent, hormone, immunosuppressive, thrombolytic, vitamin, protein, salt, desensitizer, prostaglandin, amino acid, metabolite, antiallergenic and mixtures thereof.

18. The process of claim 12, wherein the silicon hydride-containing monomer is of the general formula: 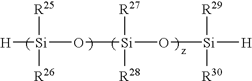
wherein z is from 0 to about 100, R²⁵, R²⁶, R²⁹, and R³⁰ independently are hydrogen, a straight or branched C₁-C₃₀ alkyl group, a C₁-C₃₀ fluoroalkyl group, a C₁-C₂₀ ester group, an ether or polyether containing group, an ureido group, an amide group, an amine group, a substituted or unsubstituted C₁-C₃₀ alkoxy group, a substituted or unsubstituted C₃-C₃₀ cycloalkyl group, a substituted or unsubstituted C₃-C₃₀ cycloalkylalkyl group, a substituted or unsubstituted C₃-C₃₀ cycloalkenyl group, a substituted or unsubstituted C₅-C₃₀ aryl group, a substituted or unsubstituted C₅-C₃₀ arylalkyl group, a substituted or unsubstituted C₅-C₃₀ heteroaryl group, a substituted or unsubstituted C₃-C₃₀ heterocyclic ring, a substituted or unsubstituted C₄-C₃₀ heterocyclolalkyl group, a substituted or unsubstituted C₆-C₃₀ heteroarylalkyl group, fluorine, a vinyl group, a C₅-C₃₀ fluoroaryl group, or a hydroxyl group, and R²⁷ and R²⁸ independently are a straight or branched C₁-C₃₀ alkyl group, a C₁-C₃₀ fluoroalkyl group, a C₁-C₂₀ ester group, an ether or polyether containing group, an ureido group, an amide group, an amine group, a substituted or unsubstituted C₁-C₃₀ alkoxy group, a substituted or unsubstituted C₃-C₃₀ cycloalkyl group, a substituted or unsubstituted C₃-C₃₀ cycloalkylalkyl group, a substituted or unsubstituted C₃-C₃₀ cycloalkenyl group, a substituted or unsubstituted C₅-C₃₀ aryl group, a substituted or unsubstituted C₅-C₃₀ arylalkyl group, a substituted or unsubstituted C₅-C₃₀ heteroaryl group, a substituted or unsubstituted C₃-C₃₀ heterocyclic ring, a substituted or unsubstituted C₄-C₃₀ heterocyclolalkyl group, a substituted or unsubstituted C₆-C₃₀ heteroarylalkyl group, fluorine, a vinyl group, a C₅-C₃₀ fluoroaryl group, or a hydroxyl group.

19. The process of claim 12, wherein the solvent in the solution is selected from the group consisting of a ketone, alcohol, ether, aliphatic hydrocarbon, aromatic hydrocarbon, sulfoxide, amide-based solvent and mixtures thereof.

20. The process of claim 12, wherein in the step of removing the polymerization product from the solution comprises removing the polymerization product from the solution and drying the polymerization product.

21. A method for treating a mammal, the method comprising administering to the mammal the matrix controlled diffusion drug delivery system of claim 1.

22. The method of claim 21, wherein the step of administering comprises:
creating an incision within an eye; and
implanting the matrix controlled diffusion drug delivery system within the eye through the incision.

23. The method of claim 21, wherein the step of administering comprises:
injecting the matrix controlled diffusion drug delivery system within an eye.

24. A method of treating an ophthalmic state, disease, disorder, injury or condition comprising administering to a mammal in need of such treatment the matrix controlled diffusion drug delivery system the drug delivery system of claim 1.

25. A method of treating an ophthalmic state, disease, disorder, injury or condition comprising administering to a mammal in need of such treatment the matrix controlled diffusion drug delivery system the drug delivery system of claim 4.
